(19) United States
(12) Patent Application Publication
(10) Pub. No.: US 2025/0262086 A1
(43) Pub. Date: Aug. 21, 2025

(54) FLUID COLLECTION ASSEMBLY INCLUDING A CUSTOMIZABLE EXTERNAL SUPPORT AND RELATED METHODS

(71) Applicant: PUREWICK CORPORATION, Covington, GA (US)

(72) Inventors: James David Hughett, Conyers, GA (US); Claire Gloeckner, Lilburn, GA (US)

(21) Appl. No.: 19/202,862

(22) Filed: May 8, 2025

Related U.S. Application Data
(63) Continuation of application No. 17/749,340, filed on May 20, 2022, now Pat. No. 12,324,767.
(60) Provisional application No. 63/192,274, filed on May 24, 2021.

(51) Int. Cl. A61F 5/451; A61F 5/44; A61M 1/00

(52) U.S. Cl. CPC A61F 5/451; A61F 5/4401; A61F 5/4405; A61M 1/802; A61F 2250/0004; A61M 2202/0496

(57) ABSTRACT

A fluid collection system may include a fluid collection device, a tube, a fluid collection container configured to receive fluid from the fluid collection device, and a vacuum source configured to pull an at least partial vacuum to draw the fluid from the fluid collection device through the tube into the fluid collection container. The fluid collection device may include a fluid impermeable barrier including an outer surface and an inner surface, the fluid impermeable barrier defining a chamber, at least one opening, and a fluid outlet. The fluid collection device may include a porous material disposed in the chamber and an external support coupled to the outer surface of the fluid impermeable barrier. The fluid impermeable barrier exhibits a first shape prior to shaping the external support and a second shape after shaping the external support with the first shape being different from the second shape.

### CROSS-REFERENCE TO RELATED APPLICATIONS

This application is a continuation of U.S. application Ser. No. 17/749,340 filed on May 20, 2022, which claims priority to U.S. Provisional Patent Application No. 63/192,274 filed on May 24, 2021, the disclosure of each of which is incorporated herein, in their entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bedpans and urinary catheters have several problems associated therewith. For example, bedpans may be prone to discomfort, spills, and other hygiene issues. Urinary catheters be may be uncomfortable, painful, and may cause urinary tract infections. Conventional fluid collection devices also may be limited to use when a patient is confined to a bed in a supine position.

Thus, users and manufacturers of fluid collection devices continue to seek new and improved devices, systems, and methods to collect fluid.

### SUMMARY

Embodiments disclosed herein are related to fluid collection assemblies and methods of using fluid collection assemblies. In an embodiment, a fluid collection assembly is disclosed. In an embodiment, a fluid collection assembly may include a fluid impermeable barrier including an outer surface and an inner surface. The fluid impermeable barrier may define a chamber, at least one opening, and a fluid outlet. The fluid collection device may further include a porous material disposed in the chamber and an external support coupled to the outer surface of the fluid impermeable barrier. The fluid impermeable barrier may exhibit a first shape prior to shaping the external support and a second shape after shaping the external support. The first shape may be different from the second shape.

In an embodiment, a fluid collection system includes a fluid collection device, a tube in fluid communication with the fluid collection device, a fluid collection container configured to receive fluid from the fluid collection device, and a vacuum source configured to pull at least a partial vacuum to draw the fluid from the fluid collection device through the tube into the fluid collection container. The fluid collection device may include a fluid impermeable barrier including an outer surface and an inner surface. The fluid impermeable barrier may define a chamber, at least one opening, and a fluid outlet. The fluid collection device may further include a porous material disposed in the chamber and an external support coupled to the outer surface of the fluid impermeable barrier. The fluid impermeable barrier may exhibit a first shape prior to shaping the external support and a second shape after shaping the external support. The first shape may be different from the second shape.

In an embodiment, a method to collect urine is disclosed. The method may include positioning a fluid collection assembly that has been shaped from a first shape to a second shape that is different from the first shape adjacent to a female urethra opening. The fluid collection assembly may include a fluid impermeable barrier including an outer surface and an inner surface, a porous material disposed in the chamber, and a moldable external support coupled mounted to the at least one outer surface of the fluid impermeable barrier. The fluid impermeable barrier may define a chamber, at least one opening, and a fluid outlet. The method may also include receiving urine from the female urethral opening into the chamber of the fluid collection assembly and applying suction with a vacuum source to draw the urine from the chamber via a tube and deposit the urine in a fluid storage container.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a block diagram of a fluid collection system, according to an embodiment.

FIG. 1B is an isomeric view of a fluid collection system, according to an embodiment.

FIG. 2A is an isometric view of a fluid collection assembly, according to an embodiment.

FIG. 2B is a cross-sectional view of the fluid collection assembly of FIG. 2A.

FIG. 3A is an isometric view of a fluid collection assembly, according to an embodiment.

FIG. 3B is an isometric view of the fluid collection assembly of FIG. 3A.

FIG. 4A is an isometric view of an external support of a fluid collection assembly, according to an embodiment.

FIG. 4B is an isometric view of the external support of FIG. 4A.

FIG. 4C is an isometric view of an external support, according to an embodiment.

FIG. 5 is a flow diagram of a method to collect urine, according to an embodiment.

### DETAILED DESCRIPTION

Embodiments disclosed herein are related to fluid collection systems and related methods. Fluid collection devices may be soiled and require frequent changes. A fluid collection assembly may be form fitting to minimize leaks and improve comfort. To reduce the constant adjustment of the fluid collection assembly, an external support may be included. In some embodiments, the external support may be shaped so that the fluid collection assembly modified to the user's specific anatomy. The fluid collection device may then be changed out and a new fluid collection device may be placed into the external support. Users and caregivers, then, are benefited from a fluid collection system that does not need to be adjusted for proper fit with each change of the device. The user may benefit from a fluid collection assembly that is customized to fit to their body and also includes a component that is reusable, washable, and would make it easier to switch from one device to the next.

In many embodiments described herein, a fluid collection system may be customized to fit the anatomy of a user. Embodiments of the fluid collection assembly may include an external support that may be modified and/or adjustable. In some embodiments, the external support may include an elastomeric material being heat adjustable. The external support may be heated and shaped and then once cooled is rigid. In some embodiments, the external support may include a semi-rigid spine and/or framework that may be bent into various shapes to have the fluid collection assembly fit the anatomy of the user. For example, the fluid collection assembly may include an external brace that allows the user to adjust the shape of the fluid collection assembly and then maintain the shape after the fluid collection device is replaced.

In some embodiments, the fluid collection system includes a fluid collection device configured to be positioned at least proximate to a urethra of a user and a tube in fluid communication with the fluid collection device. The fluid collection system may also include a fluid collection container configured to receive fluid from the fluid collection device and a vacuum source configured to pull an at least partial vacuum within the fluid collection system to draw fluid from the fluid collection device through the tube into the fluid collection container.

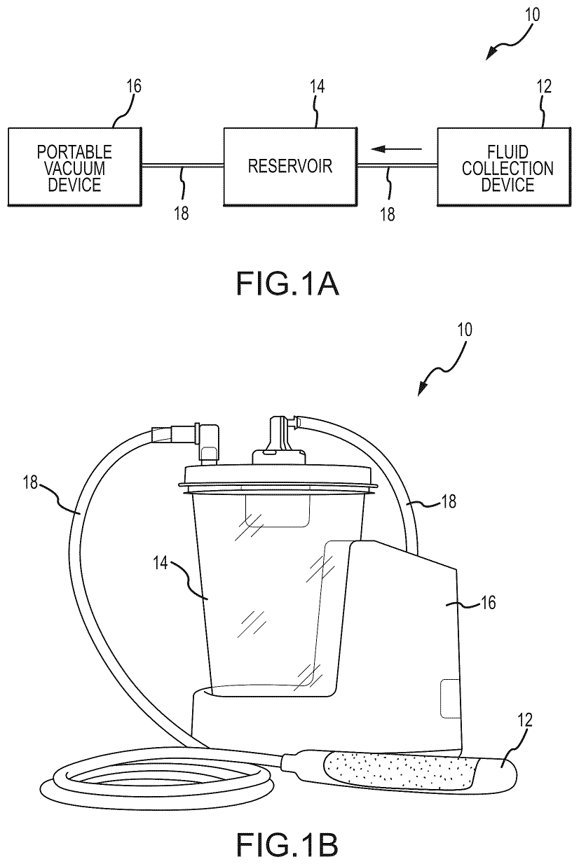

FIG. 1A is a block diagram of a fluid collection system 10 and FIG. 1B is an isomeric view of the fluid collection system 10, according to an embodiment. The fluid collection system 10 may be included in embodiments of fluid collection systems described herein. As shown in FIGS. 1A-1B, the system 10 includes a fluid (e.g., urine) collection device 12 (e.g., any of the fluid collection assemblies disclosed herein), a fluid collection container 14 (or reservoir), and a vacuum source 16 (e.g. a pump). The fluid collection device 10, the fluid collection container 14, and the vacuum source 16 may be fluidly coupled to each other via one or more tubes 18. For example, fluid collection device 10 may be operably coupled to one or more of the fluid collection container 14 or the vacuum source 16 via the tube 18. In some embodiments, the vacuum source 16 may be coupled directly to the fluid collection container 14. Fluid (e.g., urine or other bodily fluids) collected in the fluid collection device 10 may be removed from the fluid collection device 10 via the tube 18 coupled to the fluid collection device 12. Suction force may be introduced into the chamber of the fluid collection device 12 via the inlet of the tube 18 responsive to suction (e.g., vacuum) force applied at the outlet of the tube 18.

The suction force may be applied to the outlet of the tube 18 by the vacuum source 16 either directly or indirectly. The suction force may be applied indirectly via the fluid collection container 14. For example, the outlet of the tube 18 may be disposed within or fluidly coupled to an interior region of the fluid collection container 14 and an additional tube 18 may extend from the fluid collection container 14 to the vacuum source 16. Accordingly, the vacuum source 16 may apply suction to the fluid collection device 12 via the fluid collection container 14. The suction force may be applied directly via the vacuum source 16. For example, the outlet of the tube 18 may be disposed within the vacuum source 16. An additional tube 18 may extend from the vacuum source 16 to a point outside of the fluid collection device 12, such as to the fluid collection container 14. In such examples, the vacuum source 16 may be disposed between the fluid collection device 12 and the fluid collection container 14.

The vacuum source 16 may be in fluid communication with the interior region of the fluid collection container 14 and may be configured to pull at least a partial vacuum on the interior region of the fluid collection container 14 effective to draw the fluid from the fluid collection device 12 through the tube 18 into the fluid collection container 14. In some embodiments, the vacuum source 16 may be coupled directly to the fluid collection container 14, or the tube 18 may fluidly couple the vacuum source 16 with the interior region of the fluid collection container 14.

The vacuum source 16 may include one or more of a manual vacuum pump, and electric vacuum pump, a diaphragm pump, a centrifugal pump, a displacement pump, a magnetically driven pump, a peristaltic pump, or any pump configured to produce a vacuum. For example, the pump may include a diaphragm pump having a minimum pumping speed of 25 ml/second. In some embodiments, the vacuum source 16 includes a variable speed pump and/or a continuous pump. For example, the vacuum source 16 may include a variable speed pump. The vacuum source 16 may provide a vacuum or suction to remove fluid from the fluid collection device 12. In some examples, the vacuum source 16 may be powered by one or more batteries operatively coupled to the pump. In some embodiments, the battery may include a lithium ion battery. In some embodiments, the battery may be alkaline or rechargeable.

The vacuum source 16 may provide a vacuum or suction to remove fluid from the fluid collection device 12. In some examples, the vacuum source 16 may be powered by one or more of a power cord (e.g., connected to a power socket), one or more batteries, or even manual power (e.g., a hand operated vacuum pump). In some examples, the vacuum source 16 may be sized and shaped to fit outside of, on, or within the fluid collection device 12. For example, the vacuum source 16 may include one or more miniaturized pumps or one or more micro pumps. The vacuum sources disclosed herein may include one or more of a switch, a button, a plug, a remote, or any other device suitable to activate the vacuum source 16.

In some embodiments, the vacuum source 16 may be coupled directly to the fluid collection container 14. The fluid collection container 14 may be sized and shaped to retain a fluid therein. The fluid collection container 14 may include a bag (e.g., drainage bag), a bottle or cup (e.g., collection jar), or any other enclosed container for storing bodily fluid(s) such as urine. In some embodiments, the fluid collection container 14 may include a generally rigid exterior housing and an interior portion configured to contain the fluid. The fluid collection container 14 may be opaque or clear according to different embodiments and may include a generally rectangular front or rear profile or be cylindrical. The fluid collection container 14 may be reusable and dishwasher safe, and may include a generally rigid material such as polycarbonate, plastic, rubber, metal, glass, combinations thereof, or any other suitable materials.

In some examples, the tube 18 may extend from the fluid collection device 12 and attach to the fluid collection container 14 at a first point therein. The tube 18 may include a flexible tube. In some embodiments, at least a portion of the tube 18 may be substantially opaque, thereby inhibiting viewing of the fluid within the tube 18. An additional tube 18 may attach to the fluid collection container 14 at a second point thereon and may extend and attach to the vacuum source 16. Accordingly, a vacuum (e.g., suction) may be drawn through fluid collection device 12 via the fluid collection container 14. Fluid, such as urine, may be drained from the fluid collection device 12 using the vacuum source 16.

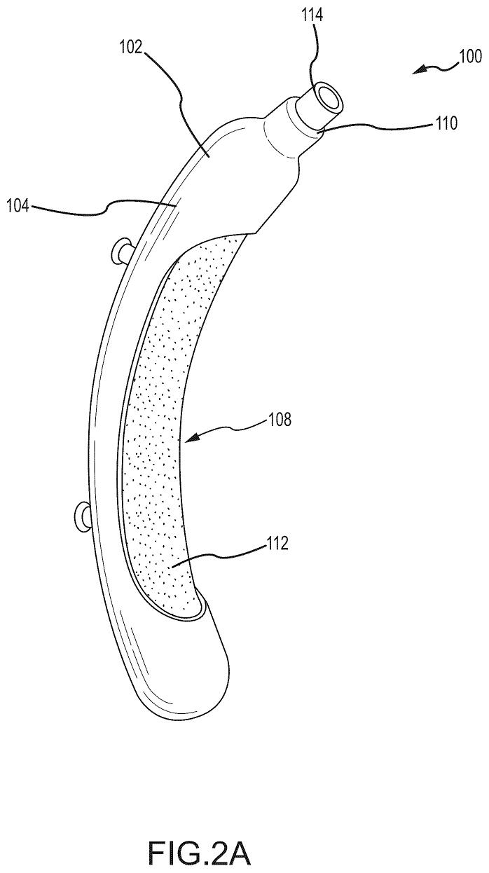

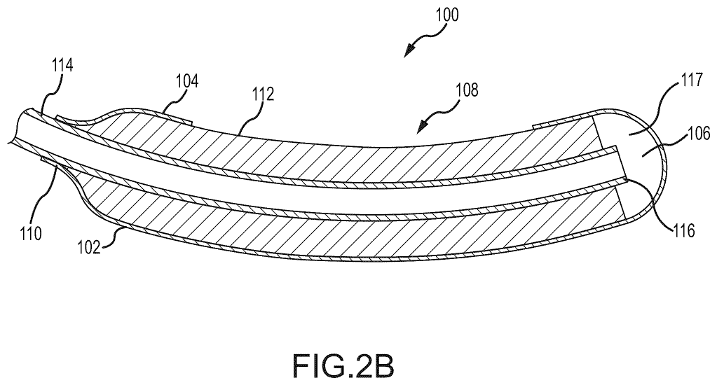

Many embodiments of fluid collection systems described herein include a fluid collection assembly having a fluid collection device configured to be disposed adjacent to a urethral opening of a user. Turning to FIG. 2A-2B, a fluid collection device 100 is shown. The fluid collection device 100 may include a fluid impermeable barrier 102 including an outer surface 104 and an inner surface, the fluid impermeable barrier defining a chamber 106, at least one opening 108, and a fluid outlet 110. A porous material 112 may be disposed in the chamber 106 within the fluid impermeable barrier 102. In some embodiments, a tube 114 may be at least partially disposed within the chamber 106.

The inner surfaces of the fluid impermeable barrier 102 at least partially defines the chamber 106 within the fluid collection device 100. The fluid impermeable barrier 102 temporarily stores the bodily fluids in the chamber 106. The fluid impermeable barrier 102 may be formed of any suitable fluid impermeable material(s), such as a fluid impermeable polymer (e.g., silicone rubber, thermoplastic polyurethane, polyolefin, polyvinyl chloride etc.), a metal film, natural latex rubber, another suitable material, or combinations thereof. As such, the fluid impermeable barrier 102 substantially prevents the bodily fluids from passing through the fluid impermeable barrier 102. In an at least one embodiment, the fluid impermeable barrier 102 may be air permeable and fluid impermeable, thus preventing leaks while allowing air flow through the chamber 106. In such an example, the fluid impermeable barrier 102 may be formed of a hydrophobic material that defines a plurality of pores. At least one or more portions of at least the outer surface 104 of the fluid impermeable barrier 102 may be formed from a soft and/or smooth material, thereby reducing chaffing.

In some examples, the fluid impermeable barrier 102 may be tubular (ignoring the opening), such as substantially cylindrical (as shown), oblong, prismatic, or flattened tubes. During use, the outer surface 104 of the fluid impermeable barrier 102 may contact the wearer. The fluid impermeable barrier 102 may be sized and shaped to fit in the gluteal cleft between the legs of a female user.

The opening 108 may provide an ingress route for fluids to enter the chamber 106. The opening 108 may be defined by the fluid impermeable barrier 102 such as by an inner edge of the fluid impermeable barrier 102. For example, the opening 108 may be formed in and extend through the fluid impermeable barrier 102, from the outer surface 104 to the inner surface, thereby enabling fluid(s) to enter the chamber 106 from outside of the fluid collection assembly 100. The opening 108 may be an elongated hole in the fluid impermeable barrier 102. For example, the opening 108 may be defined as a cutout in the fluid impermeable barrier 102. The opening 108 may be located and shaped to be positioned adjacent to a female urethra.

The fluid collection device 100 may be positioned proximate to the female urethra and bodily fluid may enter the chamber of the fluid collection assembly 100 via the opening 108. The fluid collection assembly 100 may be configured to receive the fluid(s) into the chamber 106 via the opening 108. When in use, the opening 108 may have an elongated shape that extends from a first location below the urethral opening (e.g., at or near the anus or the vaginal opening) to a second location above the urethral opening (e.g., at or near the top of the vaginal opening or the pubic hair).

The fluid collection assembly 100 includes at least one porous material 112 disposed in the chamber 106. The porous material 112 may cover at least a portion (e.g., all) of the opening 108. The porous material 112 may be exposed to the environment outside of the chamber 106 through the opening 108. The porous material 112 may be configured to wick and/or allow flow of any fluid away from the opening 104, thereby preventing the fluid from escaping the chamber 106. The permeable properties referred to herein may be wicking, capillary action, diffusion, or other similar properties or processes, and are referred to herein as “permeable” and/or “wicking.” Such “wicking” and “permeable” properties may not include absorption of fluid into the porous material 110. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the porous material 112 (e.g., absorbency), such as less than about 30 wt. % of the dry weight of the porous material 112, less than about 20 wt. %, less than about 10 wt. %, less than about 7 wt. %, less than about 5 wt. %, less than about 3 wt. %, less than about 2 wt. %, less than about 1 wt. %, or less than about 0.5 wt. % of the dry weight of the porous material 112.

In an embodiment, the porous material 112 may include at least one absorbent or adsorbent material. The porous material 112 disposed within the chamber 106 may include any material that may wick and/or allow flow of the fluid. For example, the porous material 112 may be formed from fibers from nylon (e.g., spun nylon fibers), polyester, polyethylene, polypropylene, wool, silk, linen, cotton (e.g., cotton gauze), felt, other fabrics and porous polymers, hydrophobic foam, an open cell foam polyurethane,, a coated porous material (e.g., hydrophobic coated porous material, materials with affinity to specific substances), polymeric sintered particles from polyethylene, polypropylene, polytetrafluoroethylene (PTFE), elastomeric particles, any other suitable porous materials, or combinations thereof. For example, the porous material 112 may include a body of spun nylon fibers with an outer fabric gauze layer that wraps around the body of spun nylon fibers. Forming the porous material 112 from gauze, soft fabric, and/or smooth fabric may reduce chaffing caused by the fluid collection assembly 100. In some embodiments, the porous material 112 may at least substantially and/or completely fill the portions of the chamber 106 that may not be occupied by the tube 114.

The tube 114 may be at least partially disposed in the chamber 106. The tube 114 may be used to remove fluid form the chamber 106. The tube 114 may be in fluid communication with the fluid outlet 110 of the fluid collection assembly 100. The tube 114 may include a tube inlet 116 and a tube outlet (not shown) positioned downstream from the tube inlet 116. The tube outlet may be operably coupled to a fluid collection container, such as fluid collection container 14 described in more detail above. Thus, the tube 114 may fluidly couple the chamber 106 with the fluid collection container (shown in FIG. 1B). The tube 114 may be at least partially disposed in the chamber 106. The tube 114 may be used to remove the bodily fluids from the chamber 106. The bodily fluids that are in the chamber 106 may flow through the porous material 112 to a reservoir 117. In some embodiments, the tube inlet 116 may be located in the reservoir 117. In other embodiments, the tube inlet 116 may be flush with and/or incorporated within the porous material 112 or the tube inlet 116 may be located aft of the end of the porous material 112 that partially defines the reservoir 117.

The reservoir 117 may be a substantially unoccupied portion of the chamber 106. The reservoir 117 may be defined between the fluid impermeable barrier 102 and the porous material 112. The bodily fluids that are in the chamber 106 may flow through the porous material 112 to the reservoir 117. The reservoir 117 may temporarily retain of the bodily fluids therein.

The fluid impermeable barrier 102 may retain the bodily fluids in the reservoir 117. While depicted in the distal end region in FIG. 2B, the reservoir 117 may be located in any portion of the chamber 106 such as the proximal end region, proximate the fluid outlet 110. The reservoir 117 may be located in a portion of the chamber 106 that is designed to be located in a gravimetrically low point of the fluid collection assembly when the fluid collection assembly is worn. Other embodiments of fluid collection assemblies, fluid porous materials, chambers, reservoirs, and their shapes and configurations are disclosed in U.S. Pat. Nos. 10,973,678; 10,390,989; 10,226,376; PCT Patent Application No. PCT/US2019/029608, filed on Apr. 29, 2019, the disclosure of each of which is incorporated herein, in its entirety, by this reference.

In some embodiments, the tube 114 may include a flexible material such as materials tubing (e.g., medical tubing). Such material tubing may include a thermoplastic elastomer, polyvinyl chloride, ethylene vinyl acetate, polytetrafluoroethylene, flexible metal, ceramic and composite material tubing etc. The tube 114 may include silicon or latex. In some embodiments, the tube 114 may be constructed of any suitable material. In some embodiments, the tube 114 may include one or more portions that are resilient, such as by having one or more of a diameter or wall thickness that allows the tube 114 to be flexible.

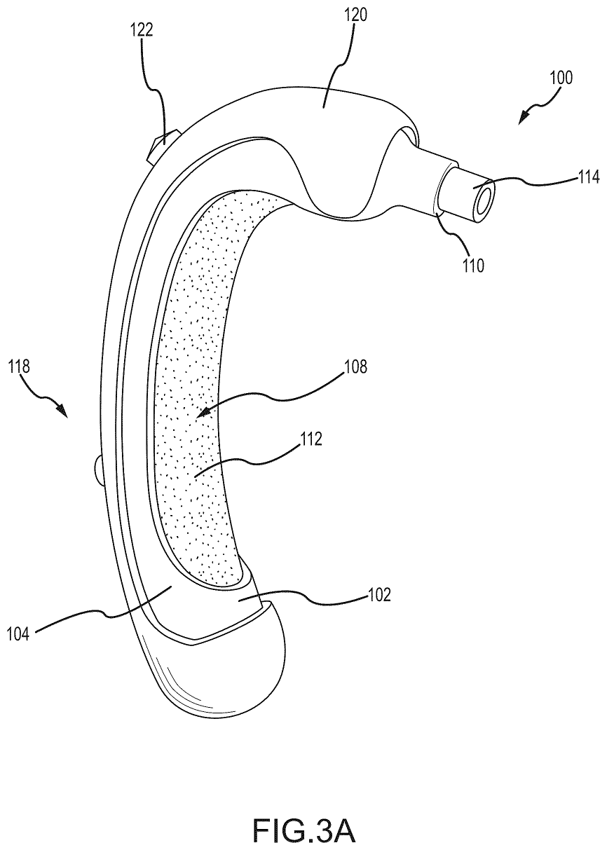

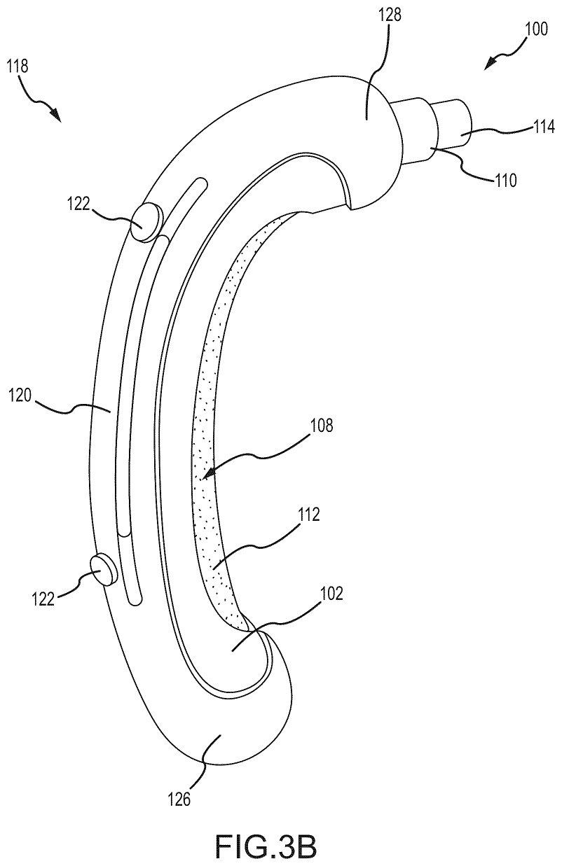

Referring now to FIG. 3A, in some embodiments, a fluid collection device 100 may be configured to be positioned at least proximate to a urethra of a user. The fluid collection device 100 may be included as a component of a fluid collection assembly 118. Generally, the fluid collection device 100 may include a surface sized to be positioned proximate or adjacent to the urethra and configured to wick urine and/or other fluids away from the user. To better shape the fluid collection device 100 to the user's anatomy, the fluid collection assembly 118 may include an external support 120 coupled to the outer surface 104 of the fluid impermeable barrier 102. The external support 120 may be shaped in some embodiments such that when coupled to the fluid collection device 100, the fluid impermeable barrier 102 may exhibit a first shape prior to shaping the external support 120 and a second shape after shaping the external support 120. The first shape may be different from the second shape. For example, prior to shaping the external support 120, the fluid impermeable barrier 102 may be generally straight and after shaping the external support 120, the fluid impermeable barrier 102 may be semi-circular or semi-elliptical. In some embodiments, the external support 120 may be configured to conform at least the porous material 112 to the anatomy of a person adjacent to a female urethral opening. As such, the fluid collection device 100 when shaped appropriately may minimize and/or prevent leakage of fluid.

In some embodiments, a portion of the fluid impermeable barrier 102 may be shaped and/or bent to fit the anatomy of the user. For example, an upper portion may be bent such that the impermeable barrier 102 generally exhibits an “L” shape. In other examples, the fluid impermeable barrier 102 may be shaped by coupling the external support 120 to the outer surface 104 such that the fluid impermeable barrier 102 generally exhibits a “C” shape. In other words, the fluid impermeable barrier 102 may include a front side that defines the at least one opening 108 and a backside opposite the front side. The external support 120 may shape the fluid impermeable barrier 102 such that at least a portion of the fluid collection assembly 118 exhibits a concave curve relative to a point above the at least one opening. To ensure that the external support 120 is able to retain the fluid collection device 100 in a predetermined shape, the external support 120 is shaped such that the fluid collection device 100 is nested within the external support 120. In some embodiments, the fluid collection device 100 may include at least one connector 122 configured to connect to the external support 120.

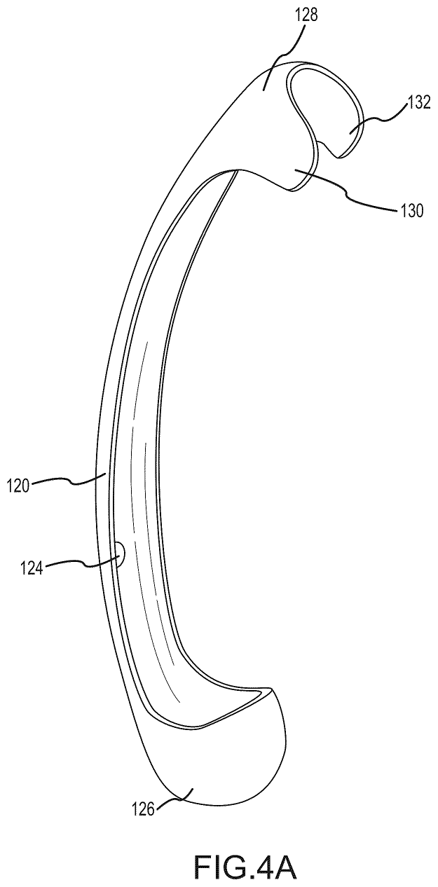

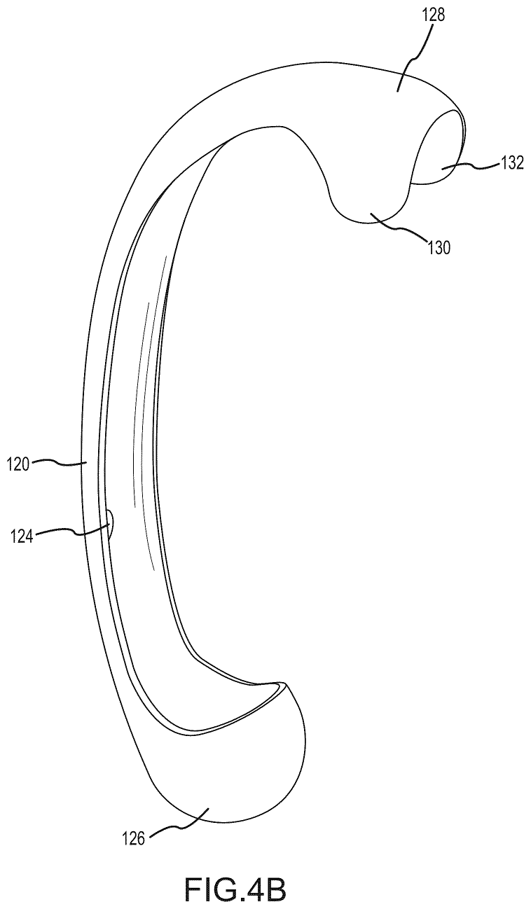

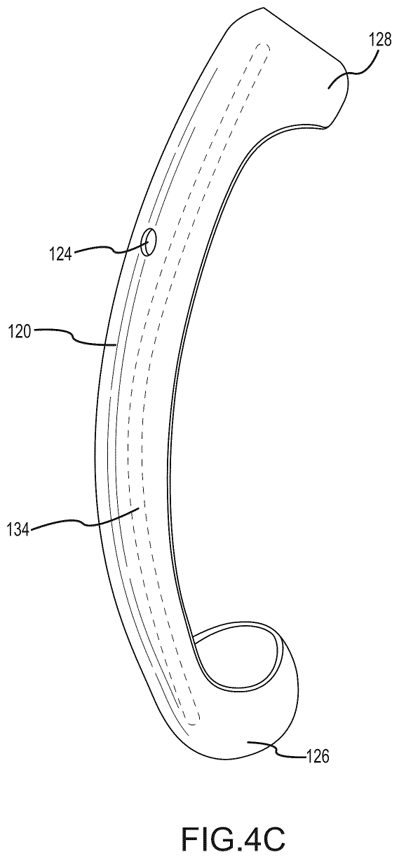

FIG. 3B illustrates the at least one connector 122 extending from the outer surface 104 of the backside of the fluid impermeable barrier 102. The external support 120 may include at least one opening 124 (as shown in FIGS. 4A-4C). The at least one connector 122 may be configured to be received by the opening 124 in the external support 120. In some embodiments, the connector 122 may couple to the opening 124 via an interference fit. In some embodiments, the connector 122 may be an adhesive. In other embodiments, the connector may be a screw, Velcro, or other suitable fastener.

Referring now to FIGS. 4A-4B, in some embodiments, the external support 120 may include an elastomeric material. The external support 120 may include at least one of ethylene vinyl acetate or a thermoplastic polyurethane. The external support 120 may include any suitable thermoplastic or blend of thermoplastics. Other suitable examples of thermoplastic materials may include polyethylene, polypropylene, polyvinyl chloride, polystyrene, polyamides, polyesters, and polyurethanes. High-temperature thermoplastics include polyether ether ketones, liquid crystalline polymers, polysulfones, and polyphenylene sulfide. In some embodiments, the external support 120 may include a polymer blend, a metal or alloy components, or any other suitable rigid or semi-rigid materials.

In some embodiments, the external support 120 may include a thermoformable material. The external support 120 may include any suitable thermoplastics that may be thermoformed. In some embodiments, the external support 120 may include Polystyrene, Polypropylene, amorphous polyethylene terephthalate (APET), Crystallized polyethylene terephthalate (CPET), and/or polyvinyl chloride (PVC). Ethylene vinyl alcohol (EVOH) may be incorporated in some embodiments; co-extrusions of the materials above may be commonly used to provide precise properties.

In some embodiments, the external support 120 may be shaped such that the fluid collection device 100 nests within the external support 120. In some embodiments, the external support 120 may include a first end 126 and a second end 128. The first end 126 may include a cup shape configured to retain the fluid collection device therein. In some embodiments, a portion of the fluid collection device 100 that does not include the fluid outlet 110 may be disposed within the first end 126 of the external support 120. In some embodiments, the second end 128 of the external support 120 may include a first arm 130 and a second arm 132 that are configured to retain the portion of the fluid collection device 100 that includes the fluid outlet 110 by way of an interference fit. The second end 128 of the external support 120 is configured to retain the fluid collection device 100 in a preferred (e.g. curved) shape without kinking or bending the tube 114. Other configurations may be suitable to shape the fluid collection assembly 118.

The external support 120 may be configured to be shaped to the anatomy of the user by the body heat of the user. For example, a user may place the fluid collection assembly 118 in a preferred configuration and keep the fluid collection assembly in contact with the user's skin to heat the external support 120 to be at, near, or above the glass transition temperature of the external support 120. The external support 120 may then form to the preferred shape and maintain the shape. In some embodiments, the external support 120 includes a glass transition temperature of about 37° C. (98° F.). In some embodiments, the external support may include an elastomeric resilience at a temperature of less than about 36° C. (97° F.). In other embodiments, the glass transition temperature may be higher than about 37° C. The glass transition temperature may be about 100° C. or less, such as about 90° C. or less, about 80° C. or less, about 70° C. or less, about 40° C. or less, or in ranges of about 30° C. to about 40° C., about 40° C. to about 60° C., about 50° C. to about 60° C., about 60° C. to about 90° C., or about 80° C. to about 100° C. In other embodiments, the glass transition temperature may be higher than about 37° C. The elastomeric resilience of the external support 120 may be about 100° C. or less, such as about 80° C. or less, about 60° C. or less, about 40° C. or less, about 30° C. or less, or in ranges of about 30° C. to about 40° C., about 40° C. to about 60° C., about 50° C. to about 60° C., about 60° C. to about 90° C., or about 80° C. to about 100° C. For example, in some embodiments, a user may place the external support 120 and/or the fluid collection assembly 118 in hot and/or boiling water (greater than 100° C.) such that the glass transition temperature is achieved. The user may then form the external support 120 to a preferred shape and allow the external support 120 to cool to below the temperature at which the external support 120 achieves an elastomeric resilience. Therefore, the external support 120 may retain the preferred shape when placed on the anatomy of the user. In some embodiments, the external support 120 may be configured to be generally straight prior to being heat formed (as shown in FIG. 4A) and concavely curved after being heat formed (as shown in FIG. 4B).

The external support 120 may include materials and/or components that assist the external support 120 in forming and/or retaining a preferred shape. Referring to FIG. 4C, the external support 120 may include a semi-rigid wire 134 or other structure such as a mesh along the length of the external support 120. In some embodiments, the semi rigid wire 134 may include copper, steel, aluminum, brass, bronze, or another suitable metal or alloy. In other embodiments, the semi-rigid wire 134 may include a polymer and/or any suitable plastic. The semi-rigid wire 134 may be monolithically formed of a single component. In some embodiments, the semi-rigid wire 134 may include more than one sections and/or elements. In some embodiments, the semi-rigid wire 134 may include a grid structure within the external support 120. The grid structure may be located within the external support 120 or coupled to an exterior surface of the external support 120. In some embodiments, the grid structure and/or the semi-rigid wire 134 may also retain the tube 114 in a predetermined shape. In some embodiments, the external support 120 may include only the grid structure.

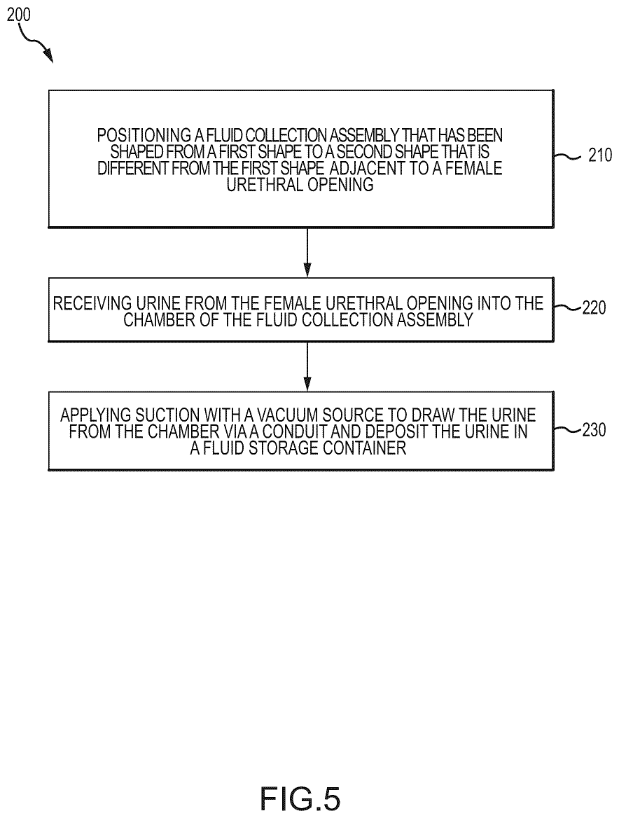

FIG. 5 is a flow diagram of a method 200 to collect urine, according to an embodiment. The method 200 includes an act 210 of positioning a fluid collection assembly that has been shaped from a first shape to a second shape that is different from the first shape adjacent to a female urethral opening. The fluid collection assembly may include a fluid impermeable barrier including an outer surface and an inner surface, the fluid impermeable barrier defining a chamber, at least one opening, and a fluid outlet. The fluid collection assembly may further include a porous material disposed in the chamber and a moldable external brace mounted to the at least one outer surface of the fluid impermeable barrier.

The method 200 also includes an act 220 receiving urine from the female urethral opening into the chamber of the fluid collection assembly. The method 200 may also include an act 230 of applying suction with a vacuum source to draw the urine from the chamber via a tube and deposit the urine in a fluid storage container.

In some embodiments, the act 210 may include a fluid collection assembly shaped from a first shape to a second shape by heating the moldable external brace to at least a glass transition temperature of the moldable external brace and causing the fluid collection assembly to conform to an anatomy of a user adjacent to the female urethral opening. In other embodiments the fluid collection assembly may be shaped from a first shape to a second shape by: heating the moldable external brace having the first shape to at least a glass transition temperature of the moldable external brace and shaping the moldable external brace to the second shape such that the fluid collection assembly conforms to an anatomy of a user adjacent to the female urethral opening. The shape may be retained by cooling the moldable external brace below the glass transition temperature. The fluid collection assembly may then be positioned within the shaped and cooled moldable external brace. In other embodiments, heating the moldable external brace to at least a glass transition temperature of the moldable external brace may include heating the moldable external brace with the body heat of the user to shape the moldable external brace. In some embodiments, act 210 may include placing the external brace adjacent to a female urethral opening where the body heat of the user heats the external brace above the glass transition temperature of the material. In some embodiments, act 210 may include heating the moldable external brace with an external heat source and then applying the heated external brace to the user to shape the moldable external brace. The external heat source may include hot water. The external brace may be placed in water heated above the glass transition temperature of the external brace and then shaped to the anatomy of the user, and then allowed to cool.

The acts of the method of collecting fluids from a user described above are for illustrative purposes. For example, the acts of the method of collecting fluids from a user can be performed in different orders, split into multiple acts, modified, supplemented, or combined. In an embodiment, one or more of the acts of the method of collecting fluids from a user can be omitted from the method. Any of the acts of the method of collecting fluids from a user can include using any of the portable fluid collection systems disclosed herein.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

What is claimed is:

1. A fluid collection assembly, comprising:
a fluid impermeable barrier defining a chamber, at least one opening, and a backside opposite the at least one opening;
an external support coupled to an outer surface of the fluid impermeable barrier, wherein the external support is complementary shaped with the backside of the fluid impermeable barrier, wherein the fluid impermeable barrier exhibits a first shape prior to shaping the external support and a second shape after shaping the external support, wherein the first shape is different from the second shape.

2. The fluid collection assembly of claim 1, wherein the external support is configured to conform the at least one opening to an anatomy of a person adjacent to a female urethral opening.

3. The fluid collection assembly of claim 1, wherein the external support includes an elastomeric material.

4. The fluid collection assembly of claim 1, wherein the fluid impermeable barrier includes a front side that defines the at least one opening and the front side is opposite the backside, wherein the external support shapes the fluid impermeable barrier such that at least a portion of the fluid collection assembly exhibits a concave curve relative to a point above the at least one opening.

5. The fluid collection assembly of claim 1, wherein the fluid impermeable barrier includes at least one connector configured to connect to the external support.

6. The fluid collection assembly of claim 5, wherein the at least one connector extends from the outer surface of the backside of the fluid impermeable barrier.

7. The fluid collection assembly of claim 1, wherein the external support is configured to be generally straight prior to being thermoformed and concavely curved after being thermoformed.

8. The fluid collection assembly of claim 3, wherein the elastomeric material includes at least one of ethylene vinyl acetate or thermoplastic polyurethane.

9. The fluid collection assembly of claim 1, wherein the external support exhibits a glass transition temperature of about 37° C. (98° F.).

10. The fluid collection assembly of claim 1, wherein the external support is configured to be shaped to an anatomy of the user by body heat of a user.

11. The fluid collection assembly of claim 1, wherein the external support exhibits an elastomeric resilience at a temperature of less than about 36° C. (97° F.).

12. The fluid collection assembly of claim 1, wherein the external support includes a semi-rigid wire along a longitudinal length of the external support.

13. An external support, comprising:
a body defining a recess shaped to receive an outer surface of a fluid collection device, wherein the external support is complementary shaped with the fluid collection device, wherein the external support comprises a flexible material such that the external support exhibits a first shape prior to shaping the external support and a second shape after shaping the external support, wherein the first shape is different than the second shape.

14. The external support of claim 13, wherein the external support comprises apertures configured to couple to least one connector disposed on the fluid collection device.

15. The external support of claim 14, wherein the at least one connector extends from the outer surface of the backside of the fluid collection device.

16. The fluid collection system of claim 13, wherein the first shape comprises a generally straight configuration and the second shape comprises a concavely curved configuration.

17. A method to collect urine, the method comprising:
positioning a fluid collection assembly that has been shaped from a first shape to a second shape that is different from the first shape adjacent to a female urethral opening, the fluid collection assembly including:
a fluid collection device defining a chamber, at least one opening, and a backside opposite the at least one opening;
a moldable external brace mounted to the at least one outer surface of the fluid collection device, wherein the moldable external brace is complementary shaped with the backside of the fluid impermeable barrier; and
receiving urine from the female urethral opening into the chamber of the fluid collection assembly.

18. The method of claim 17, wherein the fluid collection assembly is shaped from a first shape to a second shape by heating the moldable external brace to at least a glass transition temperature of the moldable external brace and causing the fluid collection assembly to conform to an anatomy of a user adjacent to the female urethral opening.

19. The method of claim 17, wherein the fluid collection assembly is shaped from a first shape to a second shape by:
heating the moldable external brace having the first shape to at least a glass transition temperature of the moldable external brace;
shaping the moldable external brace to the second shape such that the fluid collection assembly conforms to an anatomy of a user adjacent to the female urethral opening
cooling the moldable external brace below the glass transition temperature; and
positioning the fluid collection assembly within the moldable external brace.

20. The method of claim 18, wherein heating the moldable external brace to at least a glass transition temperature of the moldable external brace includes heating the moldable external brace with body heat of the user to shape the moldable external brace.

21. The method of claim 18, wherein heating the moldable external brace to at least a glass transition temperature of the moldable external brace includes heating the moldable external brace with an external heat source and applying the heated external brace to the user to shape the moldable external brace.
